(19) United States
(12) Patent Application Publication
(10) Pub. No.: US 2024/0272165 A1
(43) Pub. Date: Aug. 15, 2024

(54) A METHOD TO IDENTIFY EXCEPTIONAL ANTI-TUMOR BENEFIT FROM BRAF TARGETED THERAPIES

(71) Applicant: The Brigham and Women's Hospital, Boston, MA (US)

(72) Inventor: Hans Ragnar Widlund, Brookline, MA (US)

(21) Appl. No.: 18/568,601

(22) PCT Filed: Jun. 9, 2022
(86) PCT No.: PCT/US22/32858

§ 371 Date: Dec. 8, 2023

Related U.S. Application Data
(60) Provisional application No. 63/208,598, filed on Jun. 9, 2021.

(51) Int. Cl. G01N 33/574; A61K 31/437; A61K 31/506; A61P 35/00; C12Q 1/6886

(52) U.S. Cl. CPC G01N 33/57496; A61K 31/437; A61K 31/506; A61P 35/00; C12Q 1/6886; G01N 2800/52

(57) ABSTRACT

The present invention provides, in part, methods for identifying those subjects who are most likely to benefit from treatment with a BRAF-targeted therapy based on levels of Glyceraldehyde-3-Phosphate Dehydrogenase, Spermatogenic (GAPDHS), as well as methods for treating subjects with cancer using BRAF-targeted pharmacological inhibitors.

### CLAIM OF PRIORITY

This application claims the benefit of U.S. Provisional Application Ser. No. 63/208,598, filed on Jun. 9, 2021. The entire contents of the foregoing are incorporated herein by reference.

### TECHNICAL FIELD

The present invention provides, in part, methods for identifying those subjects who are most likely to benefit from treatment with a BRAF-targeted therapy based on levels of Glyceraldehyde-3-Phosphate Dehydrogenase, Spermatogenic (GAPDHS), as well as methods for treating subjects with cancer using BRAF-targeted pharmacological inhibitors.

### BACKGROUND

Oncogene-targeted therapies have dramatically improved the outlook and outcomes for patients, but unfortunately have not achieved the ultimate goal of durable disease remission in all patients.

### SUMMARY

Compared to standard chemotherapy, BRAF-targeted therapeutics have proven to be highly effective in extending melanoma patient survival. Almost a third of patients treated with BRAF-targeted drugs will experience durable effects and survive more than five years following treatment [5, 6]. About a third of melanoma patients treated with BRAF-targeted drugs, however, do not experience any meaningful treatment benefit—a fate that is shared, and is more frequent, in other BRAF-mutant cancers, such as papillary thyroid carcinomas and some colon and non-small cell lung carcinomas, including those carrying the same BRAF(V600E) mutation [7, 8]. Because there are now alternative treatment options for patients with advanced melanoma, identifying and understanding the mechanisms that afford these exceptional responses to BRAF(V600E)-targeted drugs would help personalize these precision medicines for improved cancer care.

Differences in treatment response may be, at least in part, the result of differences in the underlying metabolic state of not only different types of cancer, but also different tumors of the same cancer type. Evidence has begun to accumulate which indicates that the efficacy of BRAF(V600E)-targeted drugs is coupled to cancer cells' inherent programmed metabolic state. In order to drive tumorigenesis, cellular transformation requires alterations in the metabolic equilibrium in order to meet the biosynthetic demands required for rapid cellular proliferation [14]. To accommodate these requirements cells must adapt their cellular metabolic supplies, a process driven by intrinsic metabolic cues. The present inventors conceptualize that certain metabolic cues can promote oncogene-mediated transformation that subsequently prevails as a sensitive metabolic state that BRAF-targeted drugs effectively exploit. To this end, the present inventors have identified GAPDHS (spermatogenic GAPDH), whose expression level is associated mitochondrial metabolism and heightened cellular responses to BRAF inhibitors, as a potential biomarker that can inform a patient's long-term response to BRAF-targeted therapies.

Thus, provided herein are methods for determining whether a subject who has cancer is likely to benefit from treatment with a BRAF inhibitor (e.g., for predicting whether a subject will respond to treatment with a BRAF inhibitor). The methods include obtaining a sample comprising cancer cells from a subject; evaluating the presence and/or level of GAPDHS in the sample, and comparing the presence and/or level of GAPDHS with a reference level, wherein a level of GAPDHS that is greater than or equal to the reference level of GAPDHS indicates a high likelihood of response and a level of GAPDHS in a subject that is less than the reference level of GAPDHS indicates a low likelihood of response.

In some embodiments, the methods include selecting a treatment comprising administration of a BRAF inhibitor to a subject who has a level of GAPDHS that is greater than or equal to the reference level of GAPDHS.

In some embodiments, the methods include administering the treatment comprising administration of a BRAF inhibitor to a subject who has a level of GADPHS that is greater than the reference level of GAPDHS.

In some embodiments, evaluating the presence and/or level of GAPDHS in the sample comprises determining a level of GAPDHS mRNA in the sample.

In some embodiments, evaluating the presence and/or level of GAPDHS in the sample comprises determining a level of GAPDHS protein in the sample.

In some embodiments, the subject has melanoma, papillary thyroid carcinoma, colorectal carcinoma, and non-small cell lung carcinoma. In some preferred embodiments, the subject has melanoma.

Also provided herein are methods for treating a subject with cancer who has been identified as having a level of GAPDHS that is greater than or equal to the reference level of GAPDHS, comprising administering to the subject a therapeutically effective amount of a BRAF inhibitor. Also provided herein is a BRAF inhibitor for use in treating a subject with cancer who has been identified as having a level of GAPDHS that is greater than or equal to the reference level of GAPDHS.

In some embodiments of the methods and compositions described herein, the BRAF inhibitor is selected from the group consisting of BMS-908662, RO5212054 (also known as RG7256 or PLX3603), GDC-0879, PLX-4720, GSK2118436, sorafenib tosylate, LGX818, vemurafenib, dabrafenib, encorafenib, or RAF265.

In some embodiments of the methods and compositions described herein, the subject has melanoma, papillary thyroid carcinoma, colorectal carcinoma, and non-small cell lung carcinoma. In some preferred embodiments, the subject has melanoma.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. Illustration of therapeutic benefit seen in patients receiving oncogene targeted therapies aimed at BRAF(V600E) (vemurafenib) compared to classical chemotherapy.

FIG. 2. Schematic view of clinical BRAF inhibitor responses in melanoma, papillary thyroid, and colon tumors with BRAF(V600E) mutations as change in tumor size. BRAF(V600E)-mutant melanomas are more sensitive clinical BRAF inhibitor treatment compared to other cancer types with the identical mutation.

FIG. 3. Summary of intrinsic transcriptional cues that control melanoma cell metabolism and that serve to maintain a homeostatic balance between anabolic demands for proliferation and catabolic needs for energy generation FIGS. 4A-4C. There was a variable dependence on mitochondrial metabolism among melanoma cell lines and 4A-B) inhibition of mitochondrial ATP generation measured as sensitivity to 2,4-DNP protonophore (20 μg/mL), and tigecycline (mitochondrial translation inhibitor; 330 nM) in 72h growth assays, exemplifying an inverse association with 4C) energy redox status measured as NAD+/NADH ratios (NAD-NADH) during logarithmic growth.

FIGS. 5A-5B. Response to BRAF/MEK inhibition in melanoma cells was associated with the inherent metabolic state. 5A) Effects on cell doubling rates caused by vemurafenib and PD0325901 treatment in melanoma and carcinoma cell lines. Note that at 1 μM vemurafenib did not affect growth rate of the BRAF-wt cell line; SKMEL2(NRAS-Q61R), indicating on-target specificity. 5B) Pearson metric of PD-0325901 growth rate effects used on gene-level transcription data (HuExon 1.0ST) for Gene Set Enrichment Analysis (GSEA) identified specific pathways associated with sensitivity, particularly oxidative phosphorylation, and resistance to BRAF/MEK inhibition.

FIGS. 6A-6C. 6A) GAPDHS RNA levels were the most associated with sensitivity to BRAF/MEK inhibition by fold-change, and GAPDHS 6B) protein levels (Western blot assay) and 6C) enzymatic activity (GAPDH KDalert assay; Pierce/Thermo Fisher) varied according to RNA levels across melanoma cell lines.

FIGS. 7A-B. GAPDHS is a clinical marker of BRAF inhibition efficacy based on 7A) pre-treatment biopsy RNA levels from combined GSE50509 and GSE61992 (33% highest versus lowest stratifies clinical responses (**p<0.0041 with HR>3.9, based on log-rank analysis) and 7B) immunohistochemical staining of pre-treatment melanoma specimens stratify clinical response (*p<0.026 with HR>2.8 based on log-rank analysis of patient survival based on pre-treatment GAPDHS staining levels (HIGH versus LOW) across a 28 patient cohort treated with vemurafenib or combinatorial dabrafenib+trametinib).

FIGS. 8A-8C. GAPDHS is required for BRAF inhibitor sensitivity in vitro. Lentiviral spCas9/sgGAPDHS genome-editing in Malme3M 8A) reduces protein levels (WB) but not signaling consequences following vemurafenib or PD-0325901. 8B) reduction in the inherent NAD+/NADH ratio is seen, but not treatment affected changes; however the basal NADP+/NADPH ratio is higher and treatment causes divergent changes. 8C) Growth rates are less affected by vemurafenib, PD-0325901, cobimetinib, and PLX7904 due to sgGAPDHS targeting. Significance using 2-sided, 2-sample t-test; *p<0.05; **p<0.01.

FIG. 9. GAPDHS is required for BRAF inhibitor sensitivity in Malme3M xenografts (using NCR-Nu mice). Malme3M sgGAPDHS vs. sgSCR tumor xenograft treatment with dabrafenib+trametinib (n=5 for each cohort; V=d*d*1/2 mm3) normalized to V at start of treatment (100%).

FIG. 10. Schematic clinical decision matrix for treatment of patients based on presence of BRAF(V600E) mutation and baseline (prior-to-treatment) GAPDHS expression levels.

FIG. 11. Exon 2 (alt start) regulatory region of the GAPDHS gene contains three putative binding sites for NRF1 and one for MITF. Data from luciferase reporter assays in UACC62 (on GAPDHS and CYCS promoters) indicate NRF1 and PGCla cooperativity, and similarly that MITF transactivates GAPDHS dependent on the E-box (CATGTG).

FIG. 12. Commercially available ChIP antibodies against MITF (D5G7V, CST) and NRF1 (D9K6P, CST) bind the GAPDHS promoter in Malme3M cells.

FIG. 13. GAPDHS promotes BRAF(V600E)-mediated transformation (exemplified as growth factor sufficiency) of immortal melanocytes with MITF over-expression. *p<0.05 based on unpaired, 2-sided t-test.

FIGS. 14A-14B. Malme3M/sgGAPDHS 14A) OCR and 14B) ECAR measures indicate diminished mitochondrial reserve capacity and lower basal ECAR. Analysis with significance as; *p<0.05; based on 2-sided, 2-sample t-test.

### DETAILED DESCRIPTION

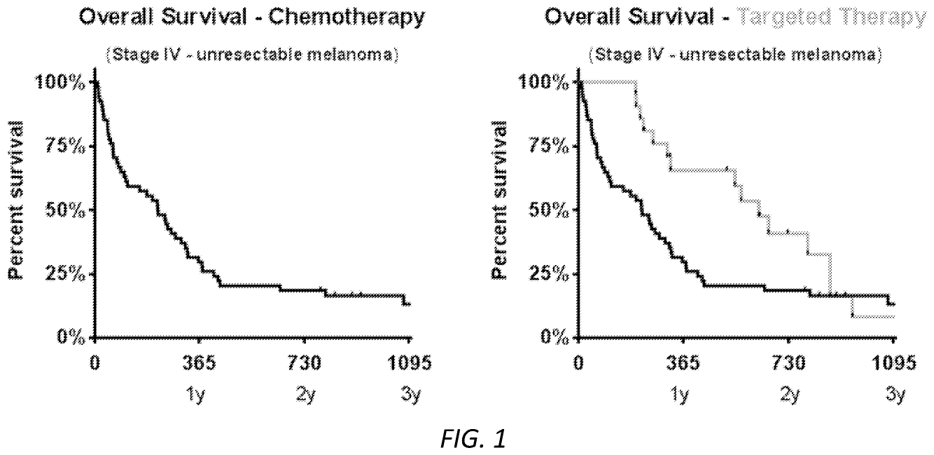

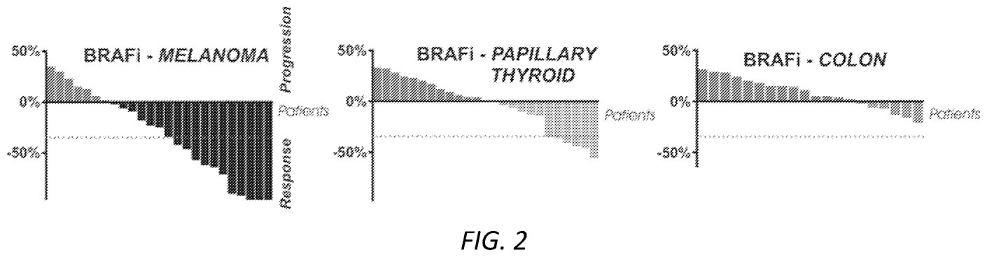

Melanoma is the most lethal form of skin cancer, compared to its two counterparts—basal cell carcinoma and squamous cell carcinoma. Resistant to traditional chemotherapies and rapid in progressing to metastasis, melanoma previously had a devastating prognosis of less than 50% survival one year after advanced state diagnosis; however, recent advancement in the development of treatment options, including BRAF inhibitors, MEK inhibitors, and immune checkpoint inhibitors, has improved outcomes and survival rates. Specifically, the first FDA approved BRAF inhibitor was vemurafenib in 2011 [10], which was shown to extend survival by 6-8 months compared to standard chemotherapy, followed by the approval of dabrafenib and encorafenib, in 2013 and 2018 respectively (illustrated in FIG. 1). Combination treatment with BRAF and MEK inhibitors have demonstrated improved patient outcomes and have been shown to improve survival benefit to an average full year [3, 4]; however, treatment with BRAF and MEK inhibitors is challenged by the fact that not all patients are afforded the same therapeutic benefit from these treatments, and presently, it is unclear who will respond to a given therapy. Specifically, not all patients whose melanomas carry the BRAF (V600E) benefit from BRAF-targeted therapeutics; yet, 30% of melanoma patients achieve enduring benefits from BRAF(V600E)-targeted drugs and go on to live for more than 5 years [5]. These variations in treatment response amongst melanoma patients, along with the marginal responses to BRAF-targeted therapy in non-melanoma BRAF(V600E)-cancers, make clear that the presence of the mutant oncogene alone is insufficient to infer efficacy (illustrated in FIG. 2). Given that there are now multiple approaches to effectively treat melanoma patients, including oncogene-targeted therapies and immune checkpoint therapies, understanding the inherent mechanisms that provide enduring clinical benefit will guide personalized care decisions by elucidating who is likely to enjoy exceptional benefit from these precision medicines.

Various mechanisms have been proposed to explain why response to BRAF-targeted therapy varies amongst cancer patients with tumors that harbor the same BRAF(V600E) mutation. Mutant RAS and BRAF oncogenes drive cellular proliferation and are causal drivers of tumorigenesis [14]; however, the increased biosynthetic demands that fuel rapid cell proliferation must be accommodated by adapting cellular metabolic supplies, which otherwise can provoke oncogene-induced senescence (OIS) caused by excessive reactive oxygen species (ROS) [reviewed in 15]. To circumvent OIS, most tumor cells turn towards accelerated glycolysis (Warburg effect) to decrease mitochondrial-derived ROS, which comes at a cost of reduced catabolic (mitochondrial) ATP production; however, an alternative means for cells to circumvent oncogene-induced metabolic stress seems to involve increasing the cellular ROS scavenging ability.

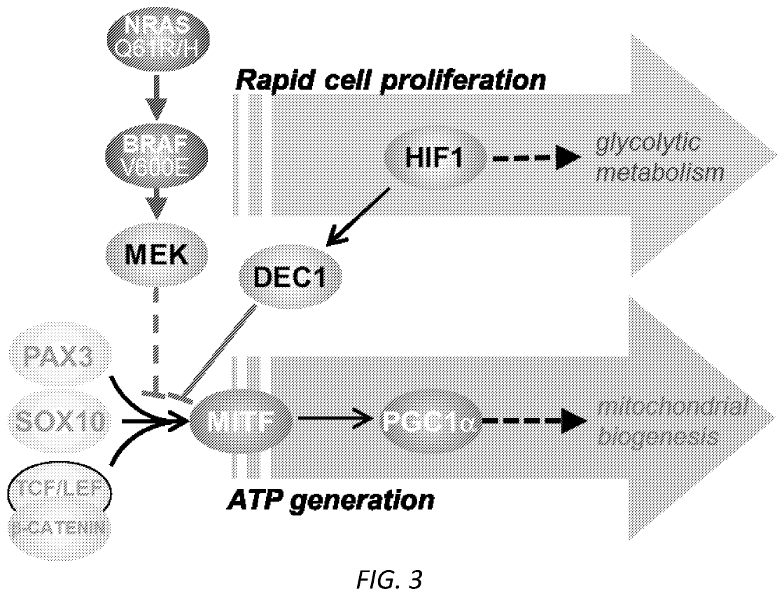

Indeed, the present inventors have previously evidenced that the melanoma-master regulator MITF cooperates with BRAF(V600E) during transformation of immortal melanocytes and that the MITF/PGC1α-axis supports ROS scavenging, suggesting that inherent cues promote resolution of oncogene-induced stress (FIG. 3) [16, 17]. Additionally, inhibition of BRAF(V600E) leads to elevated MITF/PGC1α expression that in turn increases catabolic ATP generation, indicating that oncogenic BRAF sustains the Warburg metabolic shift [18]. The present inventors have demonstrated that across melanoma cell lines there is a varied dependence on mitochondrial activity, highlighting that melanoma cells maintain a homeostatic balance between anabolic demands for cellular proliferation and catabolic needs for energy generation (illustrated in FIG. 3).

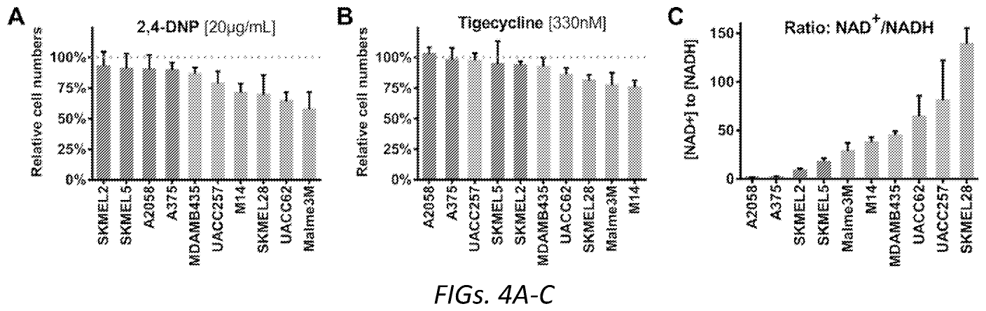

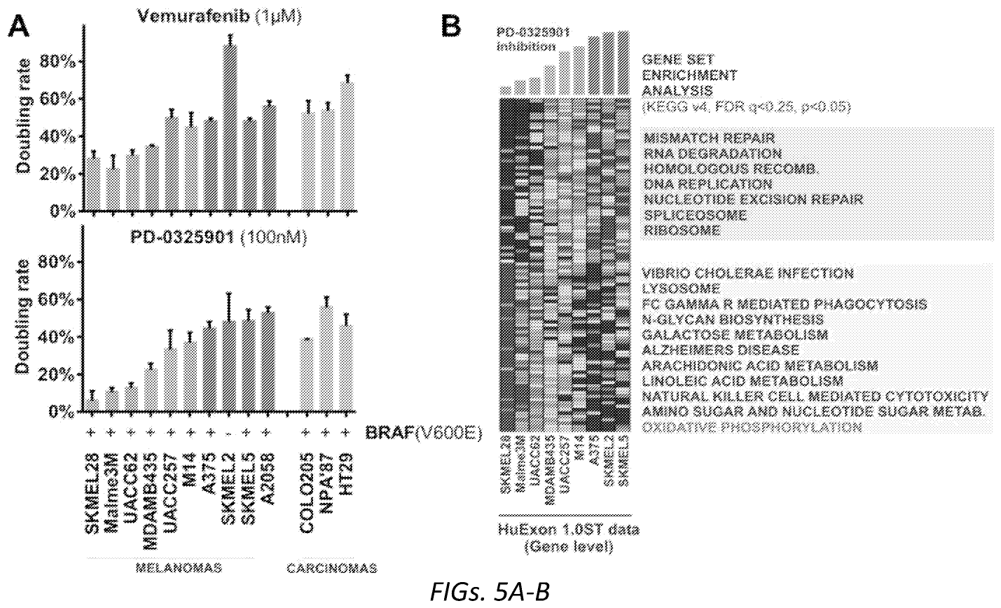

To this end, the present inventors have also found that inherent catabolic metabolism correlates with efficacy of BRAF/MEK-targeted therapies in melanoma cells (illustrated in FIG. 4). The inventors have previously shown that melanoma cells exploit a metabolic shift in response to BRAF- and MEK-targeted therapy, and when sensitivity to BRAF(V600E)- and MEK-targets agents was characterized in a cohort of melanoma cell lines, there was a high association between BRAF- and MEK-inhibitor sensitivity. While the ability of BRAF-mutations to predict sensitivity to MEK-inhibition was a previously recognized relationship [28], the inventor's data further suggests that the growth suppressive effects follow the same sensitivity gradient across cell lines. When combined with metabolic measures, the data revealed that BRAF/MEK inhibition sensitivity is inversely associated with cellular NAD+/NADH ratios, strongly indicating that the inherent metabolic state of a cell influences targeted BRAF-drug sensitivity. BRAF-sensitive melanoma cells were enriched in catabolic and cell sustaining pathways, including oxidative phosphorylation, lysosome, and amino sugar and nucleotide sugar metabolism, whereas pre-existing resistant melanoma cells were found to be enriched for anabolic RNA and DNA processes (ribosome, spliceosome, and DNA replication) required for cell division [30]. Effectively, stratification of melanoma cell lines based on their BRAF/MEK-targeted drug sensitivity displayed them as having either inherent catabolic or anabolic preferences in gene expression (illustrated in FIG. 5).

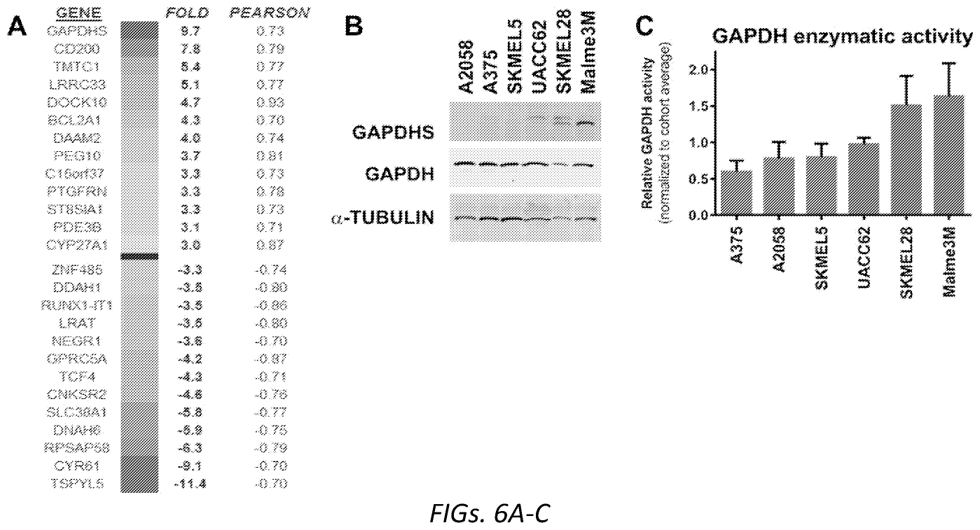

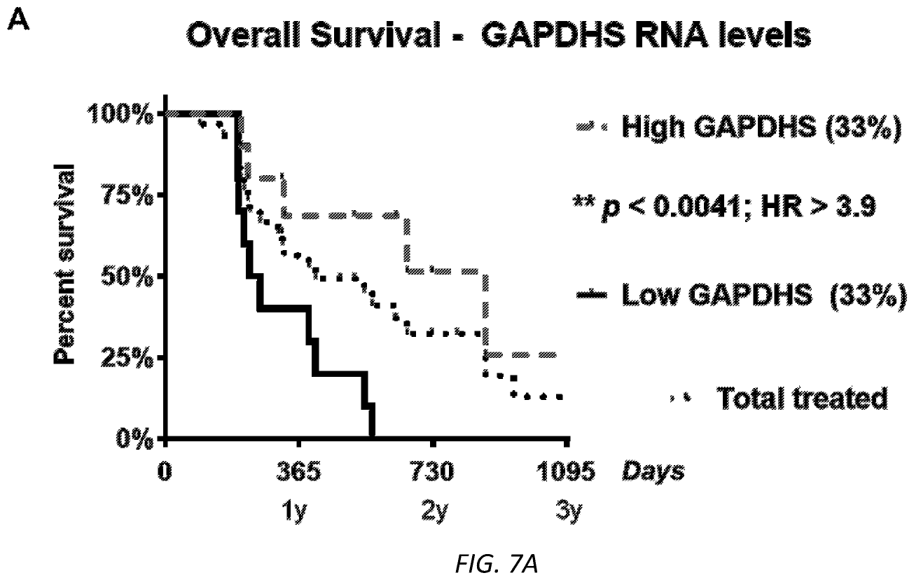

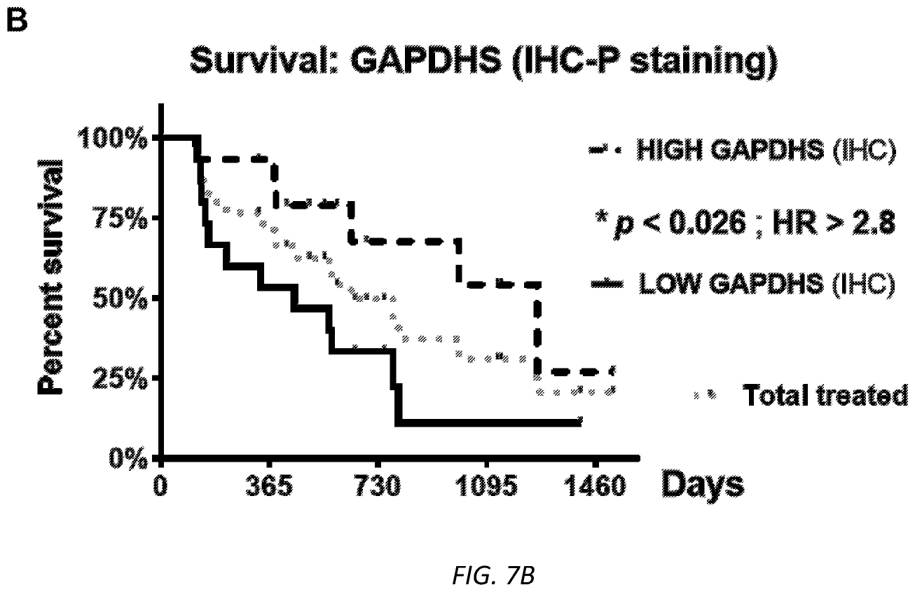

Gene expression data may prove useful in predicting cellular sensitivity or resistance to BRAF inhibitors, and may therefore have potential relevance in allowing BRAF-targeted drug responses to be predicted in pre-treatment biopsies. The present inventors have identified that spermatogenic GAPDH(S) expression associates with melanoma cell reliance on mitochondrial metabolism and exceptional responses to BRAF inhibitors (illustrated in FIGS. 6 and 7). This data indicates the prospective use of GAPDHS as a biomarker to inform patient's response prior to treatment with BRAF-targeted therapies. As a key metabolic rheostat, cellular GAPDH senses oxidative stress and its inactivation reroutes metabolic uses to favor its own reactivation by cellular detoxification pathways, which includes NADPH-generation by the oxidative-branch of the pentose phosphate pathway [11,19. Increased GAPDHS levels would consequently be expected to favor metabolic sensing but also expected to promote BRAF(V600E)-mediated transformation by circumventing metabolic stress.

As a regulator of glucose use in all organisms, the enzymatic function of GAPDH catalyzes cytoplasmic glycolysis and NADH production [19, 20]. Because NAD⁺/NADH are at an equilibrium in the cell, NAD⁺ must be generated from NADH in order for the GAPDH reaction to proceed. This can be accommodated by either mitochondrial exchange of cytoplasmic NADH (via malate-aspartate or glycerol-phosphate shuttles) or production of lactate (by lactate dehydrogenase) termed the Warburg effect [19, 38]. In addition, the reversible oxidation of a conserved catalytic cysteine in GAPDH highlights its cellular role in balancing metabolic demands with oxidative stress [39]. Specifically, reactive oxygen-species (ROS) inactivation of GAPDH reroutes glucose towards the pentose phosphate pathway (PPP) to generate NADPH. While NADPH promotes anabolic metabolism (building block synthesis, such as lipids etc), it also contributes to the cellular oxidative scavenging via the glutathione redox cycle that reactivates GAPDH [40, 41, 42].

GAPDHS enzyme shares the structure and function of somatic GAPDH but has about 3-fold higher catalytic efficiency regarding [NAD+] [43]. Hence all things equal, GAPDHS would first be saturated and setting the glycolytic rate in cells with both enzymes. However, a higher glycolytic rate would either provoke a Warburg shift involving regeneration of NAD⁺ at the expense of lactate production or increased mitochondrial function. If metabolic uses are not managed, such as by reducing reliance on oxidative phosphorylation at the cost of an enhanced glycolysis and resultant Warburg shift [44], oncogenic RAS-RAF activity can cause oncogene-induced senescence through increasing ROS. To this end, bypass of BRAF(V600E)-induced senescence was shown to involve attenuation of the pyruvate dehydrogenase complex activity [45]. An alternative means to accommodate oncogene-induced ROS would be to increase ROS scavenging (PGC1a function) with improved sensing (GAPDH function).

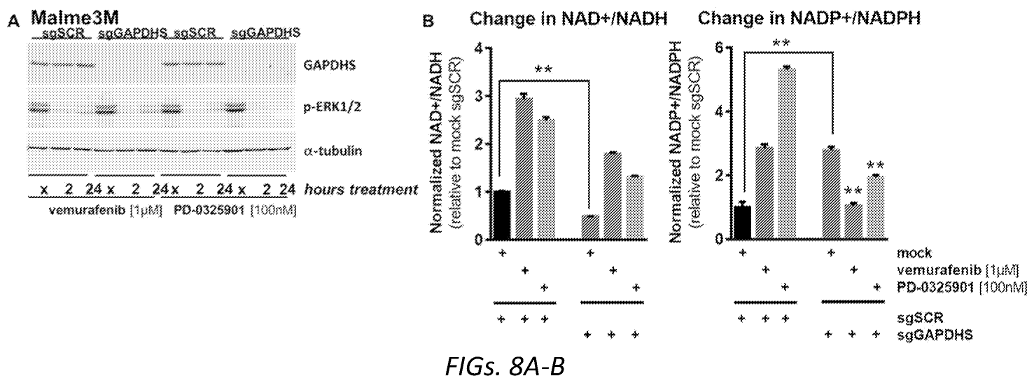

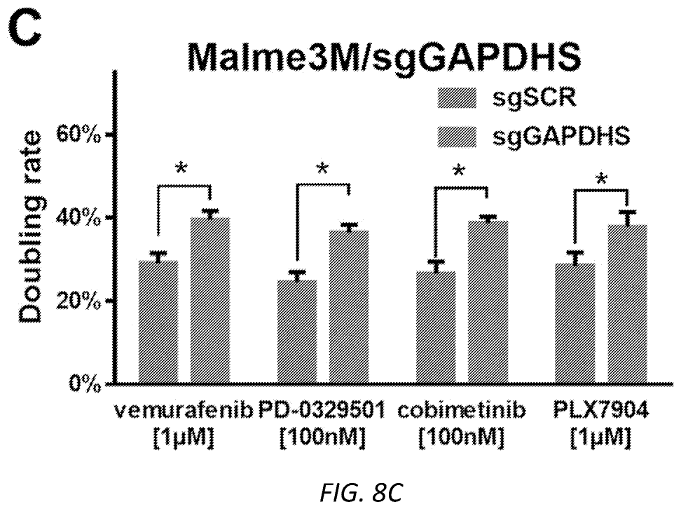

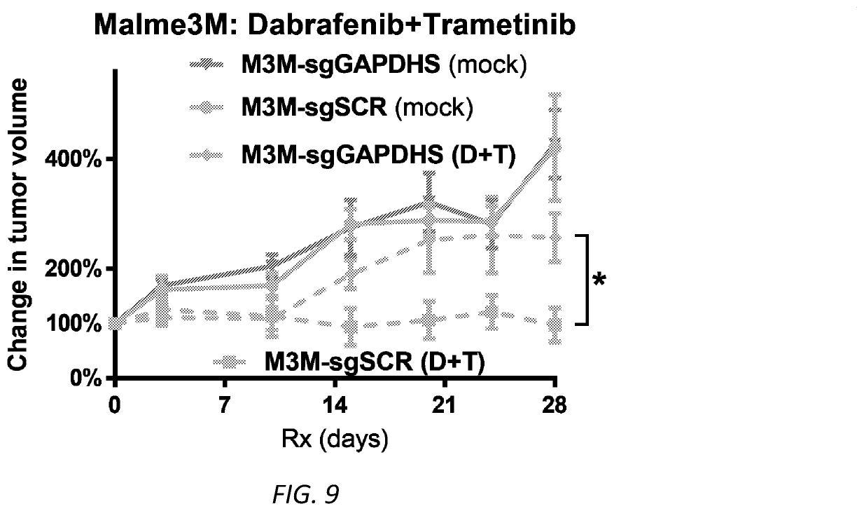

Hence, and as demonstrated herein, the present data suggest that metabolic cues afforded by GAPDHS promote BRAF(V600E) cell transformation, but yield a vulnerable state that oncogene-targeted drugs exploit, thus resulting in variable response to BRAF-targeted therapies based on GAPDHS expression (illustrated in FIGS. 8 and 9).

Thus, described herein are methods for identifying subjects who would be most likely to benefit from treatment with a BRAF inhibitors.

### Subjects

The present methods can be used in the selection and treatment of subjects with cancer, particularly those subjects with cancers harboring BRAF(V600E) mutations, e.g., melanomas, papillary thyroid carcinomas, colorectal carcinomas, and non-small cell lung carcinomas. Methods for identifying or diagnosing subjects with a cancer are known in the art, and can include biopsy, imaging, and biomarker analysis.

### Methods for Identifying and Selecting Subjects for Treatment

Described herein are methods for identifying those subjects who are most likely to benefit from treatment with a BRAF inhibitor. The methods include determining levels of GAPDHS expression (e.g., NCBI RefSeq ID NM_014364.5) in cancer cells from the subject, e.g., cancer cells obtained by biopsy, e.g., punch biopsy, needle biopsy, or tissue biopsy obtained during resection.

The methods include obtaining a sample from a subject, and evaluating the presence and/or level of GAPDHS in the sample, and comparing the presence and/or level of GAPDHS with one or more references. Gene expression of GAPDHS (e.g., mRNA levels) and protein levels (e.g., IHC protein staining) are exemplified herein, e.g., levels of soluble GAPDHS protein (e.g., NCBI RefSeq ID NP_055179.1).

Suitable reference values can be determined using methods known in the art, e.g., using standard clinical trial methodology and statistical analysis. The reference values can have any relevant form. In some cases, the reference comprises a predetermined value for a meaningful level of GAPDHS, e.g., a reference corresponding to a level of GAPDHS in a representative subject or cohort of subjects that is sensitive to BRAF inhibitors, and/or a level of GAPDHS in a representative subject or cohort of subjects that is sensitive to BRAF inhibitors.

The predetermined or reference level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with response to checkpoint inhibitors in one defined group is a fold higher, or lower, (e.g., approximately 2-fold, 4-fold, 8-fold, 16-fold or more) than the response to checkpoint inhibitors in another defined group. It can be a range, for example, where a population of subjects (e.g., control subjects) is divided equally (or unequally) into groups, such as a low-likelihood of response group and a high-likelihood of response group, or a low-likelihood of response group, a medium-likelihood of response group and a high-likelihood of response group, or into quartiles, the lowest quartile being subjects with the lowest likelihood of response and the highest quartile being subjects with the highest likelihood of response, or into n-quantiles (i.e., n regularly spaced intervals) the lowest of the n-quantiles being subjects with the lowest likelihood of response and the highest of the n-quantiles being subjects with the highest likelihood of response. As used herein, a “high” likelihood of response means that the subject is at least more likely than not to respond to a treatment.

Thus, in some cases the level of GAPDHS in a subject being greater than or equal to a reference level of GAPDHS is indicative of a high likelihood of response. In other cases the level of GAPDHS in a subject being less than or equal to the reference level of GAPDHS is indicative of a low likelihood of response.

The predetermined value can depend upon the particular population of subjects (e.g., human subjects) selected. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art.

In characterizing likelihood, or risk, numerous predetermined values can be established.

Various methods are well known within the art for the identification and/or isolation and/or purification of a biological marker, e.g., GAPDHS, from a sample. An “isolated” or “purified” biological marker is substantially free of cellular material or other contaminants from the cell or tissue source from which the biological marker is derived i.e. partially or completely altered or removed from the natural state through human intervention. For example, GAPDHS nucleic acids contained in the sample can be first isolated according to standard methods, for example using lytic enzymes, chemical solutions, or isolated by nucleic acid-binding resins following the manufacturer's instructions.

The presence and/or level of a GAPDHS nucleic acid (an exemplary sequence of human GAPDHS nucleic acid can be found in GenBank at RefSeq Acc. No. NM_014364.5) can be evaluated using methods known in the art, e.g., using polymerase chain reaction (PCR), reverse transcriptase polymerase chain reaction (RT-PCR), quantitative or semi-quantitative real-time RT-PCR, digital PCR i.e. BEAMing ((Beads, Emulsion, Amplification, Magnetics) Diehl (2006) Nat Methods 3:551-559); RNAse protection assay; Northern blot; various types of nucleic acid sequencing (Sanger, pyrosequencing, NextGeneration Sequencing); fluorescent in-situ hybridization (FISH); or gene array/chips) (Lehninger Biochemistry (Worth Publishers, Inc., current addition; Sambrook, et al, Molecular Cloning: A Laboratory Manual (3. Sup.rd Edition, 2001); Bernard (2002) Clin Chem 48(8): 1178-1185; Miranda (2010) Kidney International 78:191-199; Bianchi (2011) EMBO Mol Med 3:495-503; Taylor (2013) Front. Genet. 4:142; Yang (2014) PLOS One 9(11):e110641); Nordstrom (2000) Biotechnol. Appl. Biochem. 31(2):107-112; Ahmadian (2000) Anal Biochem 280:103-110. In some embodiments, high throughput methods, e.g., protein or gene chips as are known in the art (see, e.g., Ch. 12, Genomics, in Griffiths et al., Eds. Modern genetic Analysis, 1999,W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of GAPDHS. Measurement of the level of GAPDHS can be direct or indirect. For example, the abundance levels of GAPDHS can be directly quantitated. Alternatively, the amount of a biomarker can be determined indirectly by measuring abundance levels of GAPDHS cDNA, amplified RNAs or DNAs, or by measuring quantities or activities of RNAs, or other molecules that are indicative of the expression level of the biomarker. In some embodiments a technique suitable for the detection of alterations in the structure or sequence of nucleic acids, such as the presence of deletions, amplifications, or substitutions, can be used for the detection of biomarkers of this invention.

RT-PCR can be used to determine the expression profiles of biomarkers (U.S. Patent No. 2005/0048542A1). The first step in expression profiling by RT-PCR is the reverse transcription of the RNA template into cDNA, followed by its exponential amplification in a PCR reaction (Ausubel et al (1997) Current Protocols of Molecular Biology, John Wiley and Sons). To minimize errors and the effects of sample-to-sample variation, RT-PCR is usually performed using an internal standard, which is expressed at constant level among tissues, and is unaffected by the experimental treatment. Housekeeping genes, such actin B (ACTB (e.g., NM_001101.4)) and RPLPO (36B4, e.g., NM_001002.3), are commonly used.

Gene arrays can be prepared, e.g,. by selecting probes that comprise a polynucleotide sequence, and then immobilizing such probes to a solid support or surface. For example, the probes can comprise DNA sequences, RNA sequences, co-polymer sequences of DNA and RNA, DNA and/or RNA analogues, or combinations thereof. The probe sequences can be synthesized either enzymatically in vivo, enzymatically in vitro (e.g. by PCR), or non-enzymatically in vitro.

The presence and/or level of a GAPDHS protein can be evaluated using methods known in the art, e.g., using standard electrophoretic and quantitative immunoassay methods for proteins, including but not limited to, Western blot; enzyme linked immunosorbent assay (ELISA); biotin/avidin type assays; protein array detection; radio-immunoassay; immunohistochemistry (IHC); immune-precipitation assay; FACS (fluorescent activated cell sorting); mass spectrometry (Kim (2010) Am J Clin Pathol 134:157-162; Yasun (2012) Anal Chem 84(14):6008-6015; Brody (2010) Expert Rev Mol Diagn 10(8):1013-1022; Philips (2014) PLOS One 9(3):e90226; Pfaffe (2011) Clin Chem 57(5): 675-687). The methods typically include revealing labels such as fluorescent, chemiluminescent, radioactive, and enzymatic or dye molecules that provide a signal either directly or indirectly. As used herein, the term “label” refers to the coupling (i.e. physically linkage) of a detectable substance, such as a radioactive agent or fluorophore (e.g. phycoerythrin (PE) or indocyanine (Cy5), to an antibody or probe, as well as indirect labeling of the probe or antibody (e.g. horseradish peroxidase, HRP) by reactivity with a detectable substance.

In some embodiments, an ELISA method may be used, wherein the wells of a mictrotiter plate are coated with an antibody against which the protein is to be tested. The sample containing or suspected of containing the biological marker is then applied to the wells. After a sufficient amount of time, during which antibody-antigen complexes would have formed, the plate is washed to remove any unbound moieties, and a detectably labelled molecule is added. Again, after a sufficient period of incubation, the plate is washed to remove any excess, unbound molecules, and the presence of the labeled molecule is determined using methods known in the art. Variations of the ELISA method, such as the competitive ELISA or competition assay, and sandwich ELISA, may also be used, as these are well-known to those skilled in the art.

In some embodiments, an IHC method may be used. IHC provides a method of detecting a biological marker in situ. The presence and exact cellular location of the biological marker can be detected. Typically a sample is fixed with formalin or paraformaldehyde, embedded in paraffin, and cut into sections for staining and subsequent inspection by confocal microscopy. Current methods of IHC use either direct or indirect labelling. The sample may also be inspected by fluorescent microscopy when immunofluorescence (IF) is performed, as a variation to IHC.

Mass spectrometry, and particularly matrix-assisted laser desorption/ionization mass spectrometry (MALDI-MS) and surface-enhanced laser desorption/ionization mass spectrometry (SELDI-MS), is useful for the detection of proteins (See U.S. Pat. Nos. 5,118,937; 5,045,694; 5,719,060; 6,225,047).

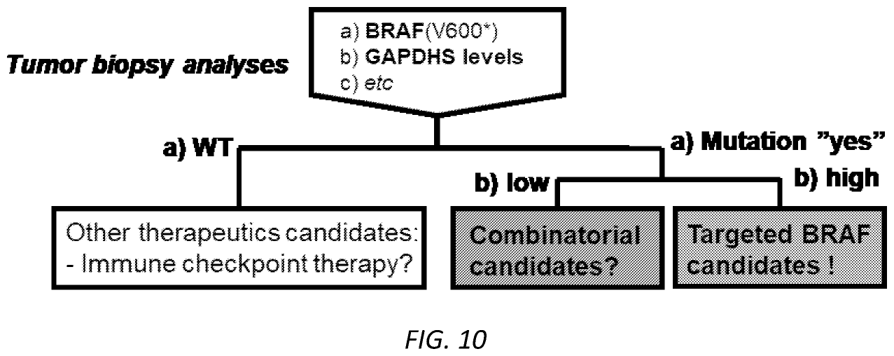

In some embodiments, the methods include selecting subjects for treatment using a BRAF inhibitor using a decision tree as shown in FIG. 10. Specifically, baseline GAPDHS levels are measured (based on measurement method, compared to a certain predetermined standard) in patient tumor biopsies, and treatment selected based on high/low GAPDHS levels; wherein high GAPDHS (GAPDHS above a reference level) indicates suitability for treatment with a BRAF inhibitor.

### BRAF Inhibitors

BRAF-targeted therapies have shown remarkable results, improving patient outcomes in certain cancers and patient groups. Currently approved BRAF inhibitors are vemurafenib, dabrafenib, and encorafenib. Additional BRAF inhibitors include BMS-908662, RO5212054 (also known as RG7256 or PLX3603), GDC-0879, PLX-4720, GSK2118436, sorafenib tosylate, LGX818, or RAF265 (see, e.g. Morris and Kopetz, F1000Prime Rep. 2013; 5:11; Proietti et al., Cancers (Basel). 2020 July; 12(7): 1823).

### Methods for Treating Subjects

The methods described herein can thus include the use of pharmaceutical compositions comprising a BRAF inhibitor, e.g., vemurafenib, and as the active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., a MEK inhibitor or an immune checkpoint inhibitor (e.g., an antibody targeting PD-1, PD-L1, CTLA-4, Lag-1, TIGIT, or Tim 3, alone or in combinations (e.g., pembrolizumab, nivolumab or ipilimumab). MEK inhibitors can include trametinib, cobimetianib, binimetinib (MEK162), selumetinib, PD-325901, CI-1040, PD035901, U0126-EtOH, PD184352 (CI-1040), TAK-733, PD98059, PD318088, BI-847325, GDC-0623, APS-2-79 HCl, Myricetin, Honokiol, SL-327, refametinib (RDEA119, Bay 86-9766), BIX 02189, BIX 02188, AZD8330, TAK-733, or Pimasertib. Pharmaceutical compositions are typically formulated to be compatible with its intended route of administration. Examples of routes of administration include parenteral, e.g., intravenous, intradermal, subcutaneous, oral or nasal (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### Examples

The invention is further described in the following examples, which do not limit the scope of the invention described in the claims.

### Example 1—Dependence on Mitochondrial Activity Across Melanoma Cell Lines

GAPDHS is thought to function by driving glycolysis through sensing the cellular redox-state, but excess oxidative stress reroutes metabolic uses to favor its own reactivation by cellular detoxification pathways, including NADPH-regeneration by the oxidative pentose phosphate pathway branch [19, 20]. By altering the metabolic stress threshold, increased GAPDHS is expected to favor metabolic sensing but also to improve BRAF(V600E)-transformation. Without wishing to be bound by theory, it is believed that the metabolic cues afforded by GAPDHS promote BRAF(V600E) cell transformation, but yield a vulnerable state that oncogene-targeted drugs, including BRAF inhibitors, exploit.

Previous work indicated that melanoma cells can be stratified by high MITF:PGC1α levels based on oxidative stress protection [17], and that BRAF- and MEK-targeted drugs activate this transcriptional axis to improve mitochondrial metabolism. To assess melanoma cells' dependence on maintaining oxidative phosphorylation, we used the proton-gradient uncoupler 2,4-DNP [21, 22] and the mitochondrial translation inhibitor tigecycline [23, 24], each of which disables catabolism-derived ATP generation [25], to assess relative NAD+/NADH ratios as a corollary to measured oxidative phosphorylation inhibition sensitivity. We found that, as expected, a high NAD+/NADH ratio indicates a catabolic phenotype, consistent with sensitivity to the mitochondrial inhibitors 2,4-DNP and tigecycline (FIG. 4). To ensure that differences in growth rates did not confound the data, we measured cell doubling times. Using Pearson correlation matrix for individual measures, an association (r=0.82; p<0.01) between 2,4-DNP and tigecycline treatment effects was obtained, and multiple measures correction revealed significant association between growth rates (doubling time) and NAD+/NADH ratios, as well as with effects of tigecycline. The inverse correlation between sensitivity to mitochondrial inhibition with growth rates and NAD+/NADH ratios highlights a homeostatic balance between anabolic demands (for proliferation) and catabolic needs (for energy generation).

### Example 2A—Examine the Relationship Between Intrinsic Catabolic Metabolism and its Correlation with the Efficacy of BRAF-Targeted Therapies

Because melanoma cells undergo metabolic shift in response to BRAF- and MEK-targeted therapy [18], we wanted to characterize the drug sensitivities across a melanoma cell line cohort (FIG. 4A). To ensure that rapidly growing cells versus slow were compared in a normalized manner, we assessed the degree of drug-mediated growth inhibition as percentage of the baseline doubling rate for each cell line (FIG. 5A). This approach was based on classical chemotherapy effect measures [26;27 recently used by others]. Despite inclusion of NRAS-mutant SKMEL2, which exhibited essentially complete BRAF inhibitor resistance (100% of normal doubling rate), we found a high association (Pearson r=0.83) between BRAF inhibitor (vemurafenib/PLX4032) and MEK-inhibitor (exemplified by PD-0325901) sensitivity. When combined with metabolic measures, our data revealed that BRAF/MEK-inhibition sensitivity is inversely associated with the cellular NAD+/NADH ratios (Pearson r=−0.74; p<0.05). Hence, these data strongly indicate that the inherent metabolic state influence targeted BRAF-drug sensitivity.

### Example 2B—Signature-Based Stratification of Clinical Response to BRAF(V600E)-Targeted Therapy

To assess whether our approach could identify authentic clinical parameters of melanoma treatment, we used signature-based stratification of published patient data. To this end, ssGSEA-based ranking [31, 32] by the resistance/sensitivity signature (Pearson r>|0.70|) at 33-percentile closeness could stratify (p<0.05) overall survival benefit within the BRAF inhibitor pre-treatment biopsy dataset [33] (FIG. 5B). Using data from biopsies obtained prior to chemotherapy (GSE22153; [34]), however, did not reveal any outcome difference. Hence, our integrative in vitro and clinical correlative analyses suggest potential relevance of using pre-treatment biopsies to predict BRAF-targeted drug responses.

### Example 2C—Examine the Relationship Between GAPDHS Expression and Clinical BRAF-Targeted Therapy Benefit

Because the combined BRAF/MEK-inhibitor sensitivity signature could stratify clinical specimens for heightened benefit from BRAF-targeted therapy, we wanted to examine its individual transcripts. The most elevated transcript within the sensitive signature was GAPDHS—the spermatogenic GAPDH. Strikingly, elevated GAPDHS expression is associated with longer overall survival (**p<0.0041) from BRAF-targeted treatments even superseding progression-free survival (PFS) in significance (*p<0.019). In the same analysis, however, the MITF/AXL ratio, which is associated with inherent chemoresistance [37], does not predict outcome (FIG. 6D).

### Example 3—Intrinsic Mechanisms Regulating GAPDHS and its Function in Metabolic Regulation

In melanoma cells, there is a close association between reliance on mitochondrial ATP-generation and sensitivity to BRAF-targeted drugs driven by expression by GAPDHS in vitro, in vivo, and within pre-treatment analyses in situ (FIGS. 8 and 9). It would therefore be advantageous to a) characterize the transcriptional regulation of GAPDHS, b) assess how GAPDHS expression promotes BRAF(V600E)-mediated transformation, c) examine how GAPDHS drives enhanced reliance on mitochondrial ATP-production and affords sensitivity to BRAF-targeted drugs by manipulating its expression in melanoma cells for in vitro and in vivo experiments, and d) assess how GAPDHS staining of pre-treatment biopsies stratifies current BRAF-targeted drug benefit.

### Example 3A—Regulation of GAPDHS Expression

Although initially identified in spermatogonia [35], expression of GAPDHS has been documented in melanoma specimens [46] and thought to be regulated by MITF [47]. Exon array data across melanoma cell lines revealed that transcription begins at exon 2, which contains an alternative start codon (ATG), omitting the sperm tethering domain, resulting in a protein that has the identical domains to GAPDH.

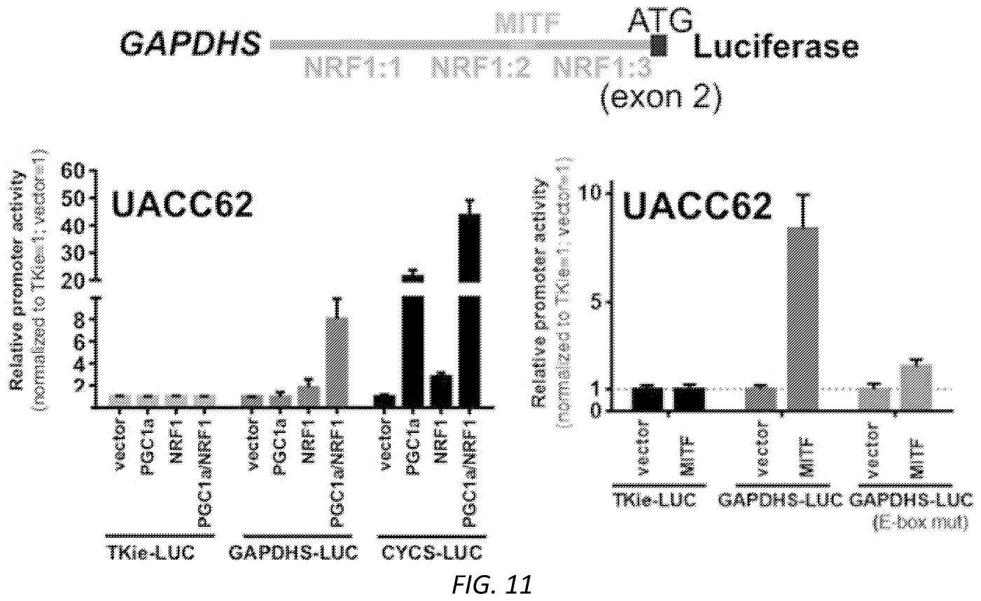

(3A-1) Metabolic cues regulate GAPDHS expression—To begin to characterize GAPDHS' transcriptional regulation, we used species homology (Ensembl) and transcription factor motif-based analysis (GenePattern/Transfac database) to identify a conserved regulatory region containing three putative NRF1 sites (1: GCGCgcGCGC (SEQ ID NO:1), 2: CaccTGCGC (SEQ ID NO:2), 3: GCcacTGCGC (SEQ ID NO:3)) and one MITF binding-site (CATGTG (SEQ ID NO:4)). We cloned this genomic region (3kbp) for use in transient dual-luciferase assays, and experiments in UACC62 indicated that NRF1 (binds DNA) and PGC1α [co-activator for NRF1: 48], synergize to activate the GAPDHS reporter akin to the cooperativity seen on cytochrome c [49, 50]. We also found that MITF transactivates the GAPDHS reporter, dependent on an intact E-box (FIG. 11). Whether MITF and/or PGC1a/NRF1 can activate the GAPDHS reporter across cancer cell lines, will be assessed using UACC62 (sensitive), A375 (resistant) melanoma cells, and HT29 (resistant) cells, in the presence or absence of MITF and NRF1 binding sites

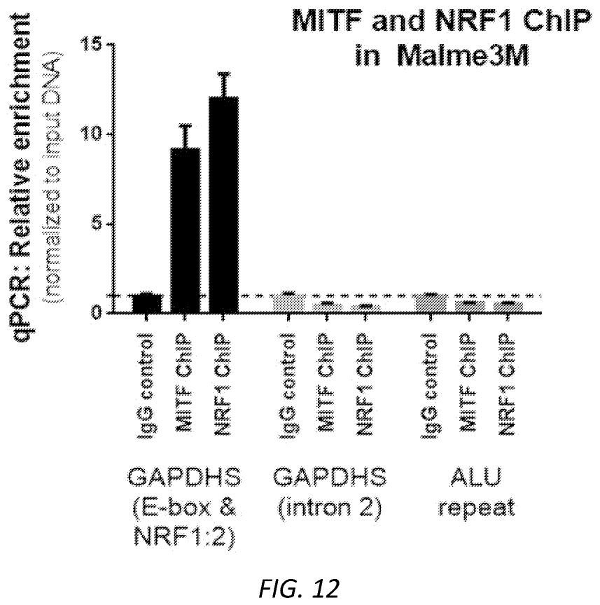

(3A-2) Transcription factors bind to GAPDHS regulatory region—Using our established chromatin immunoprecipitation (ChIP) method [51], we will assess MITF, NRF1, as well as PGC1a, binding of the GAPDHS promoter in cells. Data obtained using commercially available ChIP antibodies against MITF (D5G7V, CST) and NRF1 (D9K6P, CST) indicated that these factors bind the GAPDHS promoter in Malme3M cells (FIG. 12). In each of Malme3M, SKMEL28, and UACC62, we will use ChIP to fine-map the bona-fide regions for MITF, NRF1, as well as PGC1α [52, with Pere Puigserver using an available but commercially discontinued H-300 SCBT antibody] across the GAPDHS promoter by qPCR (amplicons every 150 bp). ChIP data will help guide reporter assays to establish which MITF, and/or PGC1a/NRF1, sites are required for activating the GAPDHS gene.

To assess site-specific requirement, we will use transient Cas9/CRISPR/template transfection and selection (using pLKO-sgRNA“site”.puro) to mutate each of the ChIP-verified MITF and NRF1 binding sites, with the expectation that these changes will reduce GAPDHS expression in cells.

### Example 3B—Assess how GAPDHS Expression Promotes BRAF(V600E)-Mediated Transformation

Since GAPDHS expression contributes to the total cellular GAPDH enzymatic activity (FIG. 6C), and its critical role in controlling cell metabolism [19], it is presumed that its' elevated activity/levels impact the metabolic state. Cellular transformation provokes cellular metabolic stress, and we have previously provided data that ectopic expression of MITF is able to facilitate BRAF(V600E)-mediated transformation of established immortal melanocytes by about 10-fold (compared to vector), which helped to elucidate the role of this genuine and amplified oncogene in the genesis of melanoma [16].

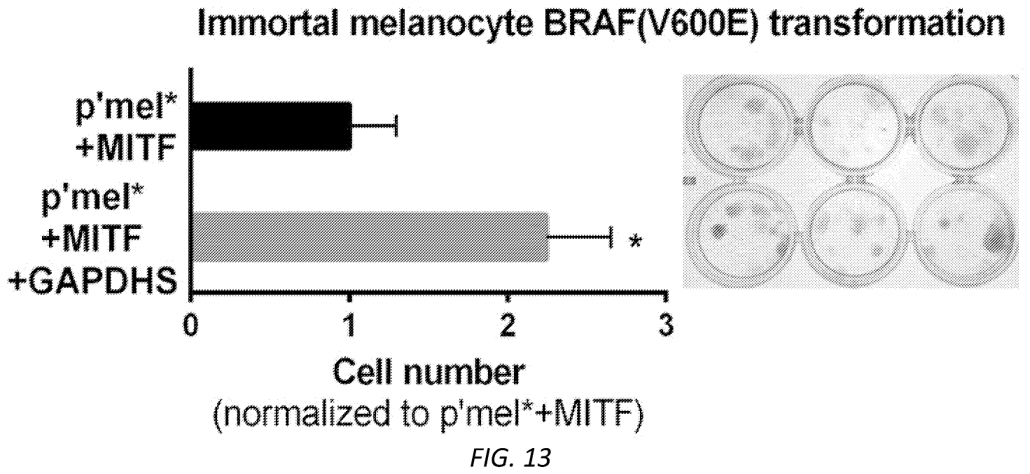

Using a human GAPDHS cDNA (ATG in exon 2) cloned into the lentiviral vector pLenti7.3 that uses GFP as a marker, we examined the effect of BRAF(V600E)-mediated transformation following ectopic expression of GAPDHS in established immortal melanocytes with over-expression of MITF (FIG. 13). Immortal melanocytes are unable to grow without adequate supplements; however, these supplements are incompatible with the presence of BRAF(V600E) (or oncogenic NRAS). Thus, subsequent to retroviral transduction of oncogenic BRAF, the melanocyte-supporting media is switched, and after extended culture (3-5 weeks), cell colonies are expected to appear and replicate. Using this methodology [16, 54], GAPDHS expression facilitated BRAF(V600E)-mediated growth factor independence in combination with MITF overexpression by about two-fold (FIG. 13).

### Example 3C—how GAPDHS Drives Mitochondrial Function and Sensitivity to BRAF-Targeted Drugs

We generated short-guide RNA (sgRNA) for CRISPR/Cas9-mediated genome-editing [60, 61] within the pLentiCRISPRv2 system [62] to assess whether GAPDHS regulates sensitivity to BRAF-targeted drugs through the metabolic state. No differences in cell signaling were detected (FIG. 8A), and sgGAPDHS cells had a lower basal NAD+/NADH ratio, with an increased basal NADP+/NADPH ratio. Furthermore, NADP+/NADPH ratios diverged upon treatment, suggesting that sgGAPDHS promoted NAPDH generation (and ROS scavenging) not seen in control cells (FIG. 8B). Strikingly, sgGAPDHS cells exhibited reduced sensitivity (measured as growth rate effect) to multiple BRAF-targeted drugs, including vemurafenib, the MEK-inhibitors PD-0325901 and cobimetinib [63], as well as the BRAF(V600E)-targeted paradox-breaker PLX7904 [64] (FIG. 8C). We did not, however, observe reduced sensitivity to BYL719 (PIK3CA inhibitor) or rapamycin (MTORC1 inhibitor) (data not shown). We will expand these analyses to also include SKMEL28 and UACC62, and assess dabrafenib and encorafenib. We will also measure basal and changes following BRAF/MEK-inhibitor on the inherent metabolic state, measured as NAD(P)+/NAD(P)H ratios and GAPDH/S activity.

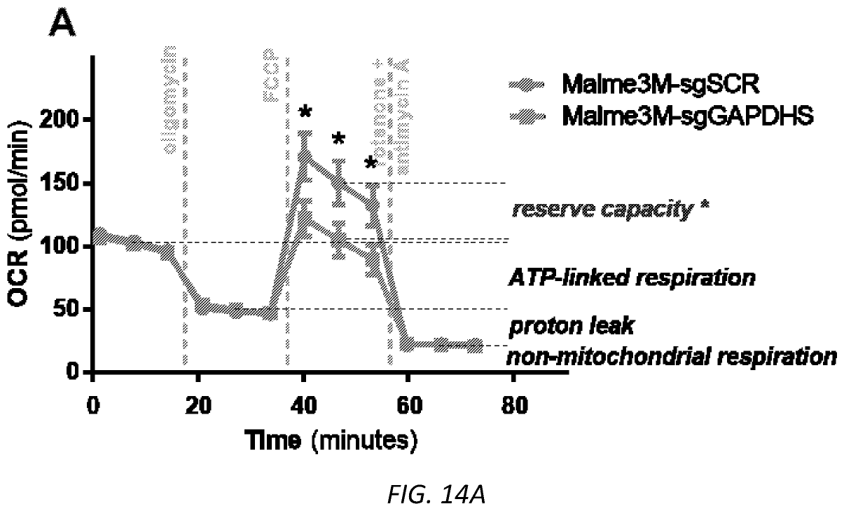

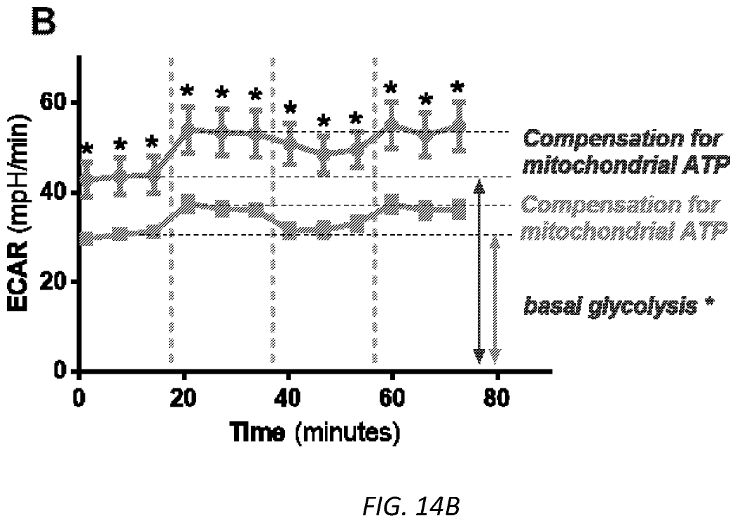

(3C-1) Respirometrics as a function of GAPDHS—We have assessed oxygen consumption (OCR) and extracellular acidification rate (ECAR) (FIG. 14) using a Seahorse FXe96 instrument [17, 65]. Using respirometric (OCR) stress tests that allow measures of basal, uncoupled (ATP-inhibited), maximal (ATP-inhibited and uncoupled), and minimum (without electron transport), preliminary analyses of Malme3M cells found that sgGAPDHS reduces basal ECAR (FIG. 14B) as expected, but without affecting the basal OCR (FIG. 14A). sgGAPDHS may offset the mitochondrial reserve capacity. Taken together, preliminary analyses indicate that GAPDHS manipulation alters BRAF-targeted drug sensitivity and alters the metabolic redox state.

(3C-2) Redox modification of GAPDHS in response to BRAF(V600E)-targeted treatment—The catalytic cysteine in GAPDHS(C224): equivalent to GAPDH(C152) is expected to be thiol-modified in response to oxidative stress caused by BRAF(V600E)-targeted drugs. Because we can detect two different mobilities of GAPDHS, performing Click-PEGylation [66] is expected to allow for improved detection and quantification of the thiol-modification levels. Click-PEGylation will be used in cells (sgGAPDHS vs sgSCR) mock or treated followed by Western blot and detection using LiCOR CLX IR scanner.

### Example 3D—GAPDHs Drives Sensitivity to BRAF-Targeted Drugs In Vivo

Pre-clinical treatment analyses are performed in melanoma tumors with manipulation of GAPDHS expression.

(3D-1) Treatment responses in xenografted melanoma tumors and effects on treatment responses—Melanoma cell lines with sgGAPDHS versus controls will be subcutaneously implanted in athymic NCR-Nu mice, and once tumors are palpable (average tumor size reaches 100 mm³), treatment will commence and be followed over time. To model clinical management, dabrafenib+trametinib will be given at a dosage of 4 mg/kg/day+0.025 mg/kg/day. Initial experiments suggest that sgGAPDHS blunts therapeutic efficacy of BRAF inhibitors in tumor xenograft models (FIG. 9).

(3D-2) Altering GAPDHS in patient-derived xenograft melanoma tumors and effects on treatment responses—Although xenografted cell lines represent the mainstay method for treatment analyses, they do not reflect the heterogeneity of genuine patient tumors. We will extend our analyses of manipulating GAPDHS expression in an orthogonally annotated cohort of melanoma PDXs (PDX-M) and follow treatment responses. The four melanoma PDX lines have high GAPDHS expression that reproducibly can be propagated in severe immunocompromised mice. To modify expression of GAPDHS in these PDX models, logarithmically growing tumor masses will be excised from euthanized mice, segregated to single cell suspensions, and subsequently transduced with sgGAPDHS lentivirus in vitro. After 24-48h culture, resulting live cells will be implanted, measured in size, and treated using trametinib+dabrafenib.

### Example 4—Assessing GAPDHS as a Biomarker of BRAF-Targeted Therapy Efficacy

Because integrated analyses identified GAPDHS expression as a potential biomarker and driver of targeted BRAF-drug sensitivity, we will extend our study to include immunohistochemical analyses. We optimized IHC(P) staining using a commercial GAPDHS antibody (goat anti-GAPDHS; R&D Systems AF6276) for detection of intensities in tissues. With antibody testing done, we conducted an initial analysis on how GAPDHS staining correlates with therapeutic benefit from BRAF-targeted inhibitors. We analyzed a cohort of 28 samples from individual patients obtained prior to treatment with either vemurafenib (n=12) or dabrafenib+trametinib combination (n=16) during clinical trials. With GAPDHS intensities in two “bins” (HIGH=strong/moderate; LOW (absent/weak), Mantel-Cox log rank testing yielded (p<0.026) stratification of treatment benefit (FIG. 7B), without effects from mono-versus combination treatment.

### REFERENCES

1. Flaherty K T, Puzanov I, Kim K B, Ribas A, McArthur G A, Sosman J A, O'Dwyer P J, Lee R J, Grippo J F, Nolop K, Chapman P B. (2010) Inhibition of mutated, activated BRAF in metastatic melanoma. N Engl J Med. 363: 809-819.
2. Chapman P B, Hauschild A, Robert C, Haanen J B, Ascierto P, Larkin J, Dummer R, Garbe C, Testori A, Maio M, Hogg D, Lorigan P, Lebbe C, Jouary T, Schadendorf D, Ribas A, O'Day S J, Sosman J A, Kirkwood J M, Eggermont A M, Dreno B, Nolop K, Li J, Nelson B, Hou J, Lee R J, Flaherty K T, McArthur G A; BRIM-3 Study Group. (2011) Improved survival with vemurafenib in melanoma with BRAF V600E mutation. N Engl J Med. 364: 2507-2516.
3. Flaherty K T, Infante J R, Daud A, Gonzalez R, Kefford R F, Sosman J, Hamid O, Schuchter L, Cebon J, Ibrahim N, Kudchadkar R, Burris H A 3rd, Falchook G, Algazi A, Lewis K, Long G V, Puzanov I, Lebowitz P, Singh A, Little S, Sun P, Allred A, Ouellet D, Kim K B, Patel K, Weber J. (2012) Combined BRAF and MEK inhibition in melanoma with BRAF V600 mutations. N Engl J Med. 367: 1694-1703.
4. Larkin J, Ascierto P A, Dreno B, Atkinson V, Liszkay G, Maio M, Mandala M, Demidov L, Stroyakovskiy D, Thomas L, de la Cruz-Merino L, Dutriaux C, Garbe C, Sovak M A, Chang I, Choong N, Hack S P, McArthur G A, Ribas A. (2014) Combined vemurafenib and cobimetinib in BRAF-mutated melanoma. N Engl J Med. 371: 1867-1876.
5. Long G V, Eroglu Z, Infante J, Patel S, Daud A, Johnson D B, Gonzalez R, Kefford R, Hamid O, Schuchter L, Cebon J, Sharfman W, McWilliams R, Sznol M, Redhu S, Gasal E, Mookerjee B, Weber J, Flaherty K T. (2017) Long-Term Outcomes in Patients With BRAF V600-Mutant Metastatic Melanoma Who Received Dabrafenib Combined With Trametinib. J Clin Oncol. 36: 667-673.
6. Robert C, Grob J J, Stroyakovskiy D, Karaszewska B, Hauschild A, Levchenko E, Chiarion Sileni V, Schachter J, Garbe C, Bondarenko I, Gogas H, Mandali M, Haanen J B A G, Lebbe C, Mackiewicz A, Rutkowski P, Nathan P D, Ribas A, Davies M A, Flaherty K T, Burgess P, Tan M, Gasal E, Voi M, Schadendorf D, Long G V. (2019) Five-Year Outcomes with Dabrafenib plus Trametinib in Metastatic Melanoma. N Engl J Med. 381: 626-636.
7. Falchook G S, Long G V, Kurzrock R, Kim K B, Arkenau T H, Brown M P, Hamid O, Infante J R, Millward M, Pavlick A C, O'Day S J, Blackman S C, Curtis C M, Lebowitz P, Ma B, Ouellet D, Kefford R F. (2012) Dabrafenib in patients with melanoma, untreated brain metastases, and other solid tumours: a phase 1 dose-escalation trial. Lancet 379: 1893-901.
8. Hyman D M, Puzanov I, Subbiah V, Faris J E, Chau I, Blay J Y, Wolf J, Raje N S, Diamond E L, Hollebecque A, Gervais R, Elez-Fernandez M E, Italiano A, Hofheinz R D, Hidalgo M, Chan E, Schuler M, Lasserre S F, Makrutzki M, Sirzen F, Veronese M L, Tabernero J, Baselga J. (2015) Vemurafenib in Multiple Nonmelanoma Cancers with BRAF V600 Mutations. N Engl J Med. 373: 726-736.
9. COSMIC database: http://cancer.sanger.ac.uk/cancergenome/projects/cosmic/
10. Puzanov I, Nathanson K L, Chapman P B, Xu X, Sosman J A, McArthur G A, Ribas A, Kim K B, Grippo J F, Flaherty K T. (2009) PLX4032, a highly selective V600EBRAF kinase inhibitor: Clinical correlation of activity with pharmacokinetic and pharmacodynamic parameters in a phase I trial. J. Clin.

Oncol. 27:15s (suppl; abstr 9021).
11. Sosman J A, Kim K B, Schuchter L, Gonzalez R, Pavlick A C, Weber J S, McArthur G A, Hutson T E, Moschos S J, Flaherty K T, Hersey P, Kefford R, Lawrence D, Puzanov I, Lewis K D, Amaravadi R K, Chmielowski B, Lawrence H J, Shyr Y, Ye F, Li J, Nolop K B, Lee R J, Joe A K, Ribas A. (2012) Survival in BRAF V600-mutant advanced melanoma treated with vemurafenib. N Engl J Med. 366: 707-714.
12. Solit D B, Rosen N. (2011) Resistance to BRAF inhibition in melanomas. N Engl J Med. 364: 772-774.
13. Robert C, Ribas A, Schachter J, Arance A, Grob J J, Mortier L, Daud A, Carlino M S, McNeil C M, Lotem M, Larkin J M G, Lorigan P, Neyns B, Blank C U, Petrella T M, Hamid O, Su S C, Krepler C, Ibrahim N, Long G V. (2019) Pembrolizumab versus ipilimumab in advanced melanoma (KEYNOTE-006): post-hoc 5-year results from an open-label, multicentre, randomised, controlled, phase 3 study. Lancet Oncol. 20: 1239-1251.
14. Samatar A A, Poulikakos P I. (2014) Targeting RAS-ERK signalling in cancer: promises and challenges. Nat Rev Drug Discov. 13: 928-942.
15. Luo C, Widlund H R, Puigserver P. (2016) PGC-1 Coactivators: Shepherding the Mitochondrial Biogenesis of Tumors. Trends Cancer 2: 619-631.
16. Garraway L A, Widlund H R, Rubin M A, Getz G, Berger A J, Ramaswamy S, Beroukhim R, Milner D A, Granter S R, Du J, Lee C, Wagner S N, Li C, Golub T R, Rimm D L, Meyerson M L, Fisher D E, Sellers W R. (2005) Integrative genomic analyses identify MITF as a lineage survival oncogene amplified in malignant melanoma. Nature 436: 117-122.
17. Vazquez F, Lim J H, Chim H, Bhalla K, Girnun G, Pierce K, Clish C B, Granter S R, Widlund H R, Spiegelman B M, Puigserver P. (2013) PGC1α expression defines a subset of human melanoma tumors with increased mitochondrial capacity and resistance to oxidative stress. Cancer Cell 23: 287-301.
18. Haq R, Shoag J, Andreu-Perez P, Yokoyama S, Edelman H, Rowe G C, Frederick D T, Hurley A D, Nellore A, Kung A L, Wargo J A, Song J S, Fisher D E, Arany Z, Widlund H R. (2013) Oncogenic BRAF regulates oxidative metabolism via PGC1α and MITF. Cancer Cell 23: 302-315.
19. Seidler N W (2013). GAPDH and intermediary metabolism. Adv Exp Med Biol. 985: 37-59
20. Yun J, Mullarky E, Lu C, Bosch K N, Kavalier A, Rivera K, Roper J, Chio I I, Giannopoulou E G, Rago C, Muley A, Asara J M, Paik J, Elemento O, Chen Z, Pappin D J, Dow L E, Papadopoulos N, Gross S S, Cantley L C. (2015) Vitamin C selectively kills KRAS and BRAF mutant colorectal cancer cells by targeting GAPDH. Science 350: 1391-1396.
21. Desquiret V, Loiseau D, Jacques C, Douay O, Malthiéry Y, Ritz P, Roussel D. (2006) Dinitrophenol-induced mitochondrial uncoupling in vivo triggers respiratory adaptation in HepG2 cells. Biochim Biophys Acta 1757: 21-30.
22. Knowles A F. (1982) Differential effects of 2,4-dinitrophenol and valinomycin (+K+) on uncoupler-stimulated ATPase of human tumor mitochondria. Biochim Biophys Acta 681: 62-71.
23. Skrtid M, Sriskanthadevan S, Jhas B, Gebbia M, Wang X, Wang Z, Hurren R, Jitkova Y, Gronda M, Maclean N, Lai C K, Eberhard Y, Bartoszko J, Spagnuolo P, Rutledge A C, Datti A, Ketela T, Moffat J, Robinson B H, Cameron J H, Wrana J, Eaves C J, Minden M D, Wang J C, Dick J E, Humphries K, Nislow C, Giaever G, Schimmer A D. (2011) Inhibition of mitochondrial translation as a therapeutic strategy for human acute myeloid leukemia. Cancer Cell 20: 674-688
24. Jenner L, Starosta A L, Terry D S, Mikolajka A, Filonava L, Yusupov M, Blanchard S C, Wilson D N, Yusupova G. (2013) Structural basis for potent inhibitory activity of the antibiotic tigecycline during protein synthesis. Proc Natl Acad Sci USA 110: 3812-3816.
25. von Ballmoos C, Wiedenmann A, Dimroth P. (2009) Essentials for ATP synthesis by FiFO ATP synthases. Annu Rev Biochem. 78: 649-672.
26. Skipper H E (1969) Improvement of the model systems. Cancer Res. 29: 2329-2333.
27. Hafner M, Niepel M, Chung M, Sorger P K. (2016) Growth rate inhibition metrics correct for confounders in measuring sensitivity to cancer drugs. Nat Methods. 13: 521-527.
28. Solit D B, Garraway L A, Pratilas C A, Sawai A, Getz G, Basso A, Ye Q, Lobo J M, She Y, Osman I, Golub T R, Sebolt-Leopold J, Sellers W R, Rosen N. (2006) BRAF mutation predicts sensitivity to MEK inhibition. Nature 439: 358-362.
29. Subramanian A, Tamayo P, Mootha V K, Mukherjee S, Ebert B L, Gillette M A, Paulovich A, Pomeroy S L, Golub T R, Lander E S, Mesirov J P. (2005) Gene set enrichment analysis: a knowledge-based approach for interpreting genome-wide expression profiles. Proc Natl Acad Sci USA. 102: 15545-15550.
30. Datta P K, Budhiraja S, Reichel R R, Jacob S T. (1997) Regulation of ribosomal RNA gene transcription during retinoic acid-induced differentiation of mouse teratocarcinoma cells. Exp Cell Res. 231: 198-205.
31. Lamb J, Crawford E D, Peck D, Modell J W, Blat I C, Wrobel M J, Lerner J, Brunet J P, Subramanian A, Ross K N, Reich M, Hieronymus H, Wei G, Armstrong S A, Haggarty S J, Clemons P A, Wei R, Carr S A, Lander E S, Golub T R.(2006) The Connectivity Map: using gene-expression signatures to connect small molecules, genes, and disease. Science 313: 1929-1935.
32. Barbie D A, Tamayo P, Boehm J S, Kim S Y, Moody S E, Dunn I F, Schinzel A C, Sandy P, Meylan E, Scholl C, Fröhling S, Chan E M, Sos M L, Michel K, Mermel C, Silver S J, Weir B A, Reiling J H, Sheng Q, Gupta P B, Wadlow R C, Le H, Hoersch S, Wittner B S, Ramaswamy S, Livingston D M, Sabatini D M, Meyerson M, Thomas R K, Lander E S, Mesirov J P, Root D E, Gilliland D G, Jacks T, Hahn W C. (2009) Systematic RNA interference reveals that oncogenic KRAS-driven cancers require TBK1. Nature 462: 108-112.
33. Rizos H, Menzies A M, Pupo G M, Carlino M S, Fung C, Hyman J, Haydu L E, Mijatov B, Becker T M, Boyd S C, Howle J, Saw R, Thompson J F, Kefford R F, Scolyer R A, Long G V. (2014) BRAF inhibitor resistance mechanisms in metastatic melanoma: spectrum and clinical impact. Clin Cancer Res. 20: 1965-1977.
34. Jonsson G, Busch C, Knappskog S, Geisler J, Miletic H, Ringner M, Lillehaug J R, Borg A, Lonning P E. (2010) Gene expression profiling-based identification of molecular subtypes in stage IV melanomas with different clinical outcome. Clin Cancer Res. 16: 3356-3367.
35. Miki K, Qu W, Goulding E H, Willis W D, Bunch D O, Strader L F, Perreault S D, Eddy EM, O'Brien DA (2004). Glyceraldehyde 3-phosphate dehydrogenase-S, a sperm-specific glycolytic enzyme, is required for sperm motility and male fertility. Proc Natl Acad Sci USA. 101: 16501-16506.
36. Long G V, Fung C, Menzies A M, Pupo G M, Carlino M S, Hyman J, Shahheydari H, Tembe V, Thompson J F, Saw R P, Howle J, Hayward N K, Johansson P, Scolyer R A, Kefford R F, Rizos H. (2014) Increased MAPK reactivation in early resistance to dabrafenib/trametinib combination therapy of BRAF-mutant metastatic melanoma. Nat Commun. 5: 5694.
37. Müller J, Krijgsman O, Tsoi J, Robert L, Hugo W, Song C, Kong X, Possik P A, Cornelissen-Steijger P D, Geukes Foppen M H, Kemper K, Goding C R, McDermott U, Blank C, Haanen J, Graeber T G, Ribas A, Lo R S, Peeper D S. (2014) Low MITF/AXL ratio predicts early resistance to multiple targeted drugs in melanoma. Nat Commun. 5: 5712.
38. Hanahan D, Weinberg R A. (2011) Hallmarks of cancer: the next generation. Cell 144: 646-674.
39. Peralta D, Bronowska A K, Morgan B, Dóka É, Van Laer K, Nagy P, Grater F, Dick T P (2015). A proton relay enhances H₂O₂ sensitivity of GAPDH to facilitate metabolic adaptation. Nat Chem Biol. 11: 156-163.
40. Padgett C M, Whorton A R. (1997) Glutathione redox cycle regulates nitric oxide-mediated glyceraldehyde-3-phosphate dehydrogenase inhibition. Am J Physiol. 272: C99-108.
41. Shenton D, Grant C M. (2003) Protein S-thiolation targets glycolysis and protein synthesis in response to oxidative stress in the yeast Saccharomyces cerevisiae. Biochem J. 374: 513-519.
42. Hwang N R, Yim S H, Kim Y M, Jeong J, Song E J, Lee Y, Lee J H, Choi S, Lee K J. (2009) Oxidative modifications of glyceraldehyde-3-phosphate dehydrogenase play a key role in its multiple cellular functions. Biochem J. 423: 253-264.
43. Chaikuad A, Shafqat N, Al-Mokhtar R, Cameron G, Clarke A R, Brady R L, Oppermann U, Frayne J, Yue W W (2011). Structure and kinetic characterization of human sperm-specific glyceraldehyde-3-phosphate dehydrogenase, GAPDS. Biochem J. 435: 401-409.
44. Lee A C, Fenster B E, Ito H, Takeda K, Bae N S, Hirai T, Yu Z X, Ferrans V J, Howard B H, Finkel T. (1999) Ras proteins induce senescence by altering the intracellular levels of reactive oxygen species. J Biol Chem. 274: 7936-7940.
45. Kaplon J, Zheng L, Meissl K, Chaneton B, Selivanov V A, Mackay G, van der Burg S H, Verdegaal E M, Cascante M, Shlomi T, Gottlieb E, Peeper D S. (2013) A key role for mitochondrial gatekeeper pyruvate dehydrogenase in oncogene-induced senescence. Nature 498: 109-112.
46. Falkenius J, Lundeberg J, Johansson H, Tuominen R, Frostvik-Stolt M, Hansson J, Egyhazi Brage S. (2013) High expression of glycolytic and pigment proteins is associated with worse clinical outcome in stage III melanoma. Melanoma Res. 23: 452-460.
47. Hoek K S, Schlegel N C, Eichhoff O M, Widmer D S, Praetorius C, Einarsson S O, Valgeirsdottir S, Bergsteinsdottir K, Schepsky A, Dummer R, Steingrimsson E. (2008) Novel MITF targets identified using a two-step DNA microarray strategy. Pigment Cell Melanoma Res. 21: 665-676.
48. Wu Z, Puigserver P, Andersson U, Zhang C, Adelmant G, Mootha V, Troy A, Cinti S, Lowell B, Scarpulla R C, Spiegelman BM. (1999) Mechanisms controlling mitochondrial biogenesis and respiration through the thermogenic coactivator PGC-1. Cell 98: 115-124.
49. Puigserver P, Wu Z, Park C W, Graves R, Wright M, Spiegelman BM. (1998) A cold-inducible coactivator of nuclear receptors linked to adaptive thermogenesis. Cell 92: 829-839.
50. Evans M J, Scarpulla R C. (1989) Interaction of nuclear factors with multiple sites in the somatic cytochrome c promoter. Characterization of upstream NRF-1, ATF, and intron Sp1 recognition sequences. J Biol Chem. 264: 14361-14368.
51. Widlund H R, Horstmann M A, Price E R, Cui J, Lessnick S L, Wu M, He X, Fisher D E. (2002) Beta-catenin-induced melanoma growth requires the downstream target Microphthalmia-associated transcription factor. J Cell Biol. 158: 1079-1087.
52. Luo C, Lim J H, Lee Y, Granter S R, Thomas A, Vazquez F, Widlund H R, Puigserver P. (2016) A PGC1α-mediated transcriptional axis suppresses melanoma metastasis. Nature 537:422-426.
53. Langmead B, Salzberg S L. (2012) Fast gapped-read alignment with Bowtie 2. Nat Methods. 9: 357-359.
54. Jane-Valbuena J, Widlund H R, Perner S, Johnson L A, Dibner A C, Lin W M, Baker A C, Nazarian R M, Vijayendran K G, Sellers W R, Hahn W C, Duncan L M, Rubin M A, Fisher D E, Levi A Garraway L A. (2010) An Oncogenic Role for ETV1 in Melanoma, Cancer Res 70: 2075-2084.
55. Sirover M A. (2018) Pleiotropic effects of moonlighting glyceraldehyde-3-phosphate dehydrogenase (GAPDH) in cancer progression, invasiveness, and metastases. Cancer Metastasis Rev. 37: 665-676..
56. Boradia V M, Manoj Raje M, Raje C I. (2014) Protein Moonlighting in Iron Metabolism: glyceraldehyde-3-phosphate Dehydrogenase (GAPDH), Biochem Soc Trans 42: 1796-1801.
57. Kim D, Langmead B, Salzberg S L. (2015) HISAT: a fast spliced aligner with low memory requirements. Nat Methods. 12: 357-360.
58. Hall A, Meyle K D, Lange M K, Klima M, Sanderhoff M, Dahl C, Abildgaard C, Thorup K, Moghimi S M, Jensen P B, Bartek J, Guldberg P, Christensen C. (2013) Dysfunctional oxidative phosphorylation makes malignant melanoma cells addicted to glycolysis driven by the (V600E)BRAF oncogene. Oncotarget 4: 584-599.
59. Dankort D, Curley D P, Cartlidge R A, Nelson B, Karnezis A N, Damsky W E Jr, You M J, DePinho R A, McMahon M, Bosenberg M. (2009) Braf(V600E) cooperates with Pten loss to induce metastatic melanoma. Nat Genet. 41: 544-552
60. Jinek M, Chylinski K, Fonfara I, Hauer M, Doudna J A, Charpentier E (2012). A programmable dual-RNA-guided DNA endonuclease in adaptive bacterial immunity. Science 337: 816-821.
61. Sander J D, Joung J K (2014). CRISPR-Cas systems for editing, regulating and targeting genomes. Nat Biotechnol. 32: 347-355.
62. Sanjana N E, Shalem O, Zhang F (2014). Improved vectors and genome-wide libraries for CRISPR screening. Nat Methods. 11: 783-784.
63. Hoeflich K P, Merchant M, Orr C, Chan J, Den Otter D, Berry L, Kasman I, Koeppen H, Rice K, Yang N Y, Engst S, Johnston S, Friedman L S, Belvin M (2012). Intermittent administration of MEK inhibitor GDC-0973 plus PI3K inhibitor GDC-0941 triggers robust apoptosis and tumor growth inhibition. Cancer Res. 72: 210-219.
64. Zhang C, Spevak W, Zhang Y, Burton E A, Ma Y, Habets G, Zhang J, Lin J, Ewing T, Matusow B, Tsang G, Marimuthu A, Cho H, Wu G, Wang W, Fong D, Nguyen H, Shi S, Womack P, Nespi M, Shellooe R, Carias H, Powell B, Light E, Sanftner L, Walters J, Tsai J, West B L, Visor G, Rezaei H, Lin P S, Nolop K, Ibrahim P N, Hirth P, Bollag G (2015). RAF inhibitors that evade paradoxical MAPK pathway activation. Nature 526: 583-586.
65. Luo C, Lim J H, Lee Y, Granter S R, Thomas A, Vazquez F, Widlund H R, Puigserver P (2016). A PGC1α-mediated transcriptional axis suppresses melanoma metastasis. Nature 537: 422-426.
66. van Leeuwen L A G, Hinchy E C, Murphy M P, Robb E L, Cochemé H C. (2017) Click-PEGylation—A Mobility Shift Approach to Assess the Redox State of Cysteines in Candidate Proteins. Free Radic Biol Med. 108: 374-382.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for determining whether a subject who has cancer is likely to benefit from treatment with a BRAF inhibitor, the method comprising:
providing a sample comprising cancer cells from a subject;
evaluating presence and/or level of GAPDHS in the sample, and
comparing the presence and/or level of GAPDHS with a reference level, wherein a level of GAPDHS that is greater than or equal to the reference level of GAPDHS indicates a high likelihood of response and a level of GAPDHS in a subject that is less than the reference level of GAPDHS indicates a low likelihood of response.

2. The method of claim 1, wherein the sample comprising cancer cells is obtained by punch biopsy, needle biopsy, or tissue biopsy obtained during resection.

3. The method of claim 1, further comprising selecting a treatment comprising administration of a BRAF inhibitor to a subject who has a level of GAPDHS that is greater than or equal to the reference level of GAPDHS.

4. The method of claim 3, further comprising administering the treatment comprising administration of a BRAF to a subject who has a level of GAPDHS that is greater than or equal to the reference level of GAPDHS.

5. The method of claim 1, wherein evaluating the presence and/or level of GAPDHS in the sample comprises determining a level of GAPDHS mRNA in the sample.

6. The method of claim 1, wherein evaluating the presence and/or level of GAPDHS in the sample comprises determining a level of GAPDHS protein in the sample.

7. The method of claim 1, wherein the subject has melanoma, papillary thyroid carcinoma, colorectal carcinoma, or non-small cell lung carcinoma.

8. The method of claim 7, wherein the subject has melanoma.

9. A method for treating a subject with cancer, the method comprising administering to the subject a therapeutically effective amount of a BRAF inhibitor.

10. The method of claim 1, wherein the BRAF inhibitor is selected from the group consisting of BMS-908662, RO5212054, GDC-0879, PLX-4720, GSK2118436, sorafenib tosylate, LGX818, vemurafenib, dabrafenib, encorafenib, or RAF265.

11. A method for treating a subject who has cancer, the method comprising:
providing a sample comprising cancer cells from a subject;
evaluating presence and/or level of GAPDHS in the sample,
comparing the presence and/or level of GAPDHS with a reference level, identifying a subject who has a level of GAPDHS that is greater than or equal to the reference level of GAPDHS, and
administering to the subject a treatment comprising administration of a BRAF inhibitor.

12. The method of claim 11, wherein the sample comprising cancer cells is obtained by punch biopsy, needle biopsy, or tissue biopsy obtained during resection.

13. The method of claim 11, wherein evaluating the presence and/or level of GAPDHS in the sample comprises determining a level of GAPDHS mRNA in the sample.

14. The method of claim 11, wherein evaluating the presence and/or level of GAPDHS in the sample comprises determining a level of GAPDHS protein in the sample.

15. The method of claim 11, wherein the subject has melanoma, papillary thyroid carcinoma, colorectal carcinoma, or non-small cell lung carcinoma.

16. The method of claim 11, wherein the subject has melanoma.

17. The method of claim 11, wherein the BRAF inhibitor is selected from the group consisting ofBMS-908662, RO5212054, GDC-0879, PLX-4720, GSK2118436, sorafenib tosylate, LGX818, vemurafenib, dabrafenib, encorafenib, or RAF265.
